(19) United States
(12) Patent Application Publication
(10) Pub. No.: US 2026/0360503 A1
(43) Pub. Date: Nov. 12, 2026

(54) Computer Application for Health Behavior Goal Selection, Monitoring, and Recommendations

(71) Applicant: Google LLC, Mountain View, CA (US)

(72) Inventors: Naghmeh Rezaei, Redwood City, CA (US); Hulya Emir-Farinas, Alameda, CA (US); Qian He, Hayward, CA (US)

(21) Appl. No.: 19/139,210

(22) PCT Filed: Dec. 22, 2022
(86) PCT No.: PCT/US2022/053876

§ 371 Date: Jun. 13, 2025

(51) Int. Cl. G16H 20/30; G16H 40/67; G16H 50/20

(52) U.S. Cl. CPC G16H 20/30; G16H 40/67; G16H 50/20

(57) ABSTRACT

A computer-implemented method includes monitoring one or more metrics of physiological data of a user based at least in part on receipt of input data indicative of a health behavior goal selected or defined by the user. The one or more metrics are associated with the health behavior goal. The method also includes tracking adherence to the health behavior goal by the user over a defined time. Further, the method includes detecting a statistical change to at least one metric value of the one or more metrics based at least in part on adherence to the health behavior goal by the user over the defined time. Moreover, the method includes performing one or more operations based at least in part on detecting the statistical change.

### FIELD

The present disclosure relates generally to a computer application implemented on a wearable device, external computing device, and/or server system that allows a user to select one or more health behavior goals, monitor metrics relating to said goals, and receive recommendations relating to said goals.

### BACKGROUND

Individuals are unique and their motivational and adherence patterns in striving for a behavior goal can differ significantly. Health-related changes in response to a behavior change can also vary between people. The idea of self-experimentation is not new but the technology that enables it is relatively recent and advances in sensors and wearable technologies have made it increasingly possible for individuals to collect data about themselves with the goal of self-knowledge through personal data. However, gaining self-knowledge can be more challenging than only a simple task of data collection.

For example, the human body can produce biological signals that are non-stationary and stochastic in nature. Hence, it is natural for sensor-measured signals to fluctuate. These natural variations are not limited to biological signals. Rather, human behavior can also vary over time and even day-to-day due to personal or environmental factors as well as seasonal effects. Examples of these biological signals and health-related behaviors (health behaviors) may be, for example, daily resting heart rate, sleep duration, step counts, and bedtime.

Due to these natural variations, meaningful changes in biological signals or behaviors might not be easily recognizable. In the context of self-experimentation, for example, individuals might not recognize changes, e.g., when the values are small relative to variability of corresponding signals.

Accordingly, the present disclosure is directed to a computer application that can be implemented on a wearable device, external computing device, and/or server system to allow a user to select one or more health behavior goals. Thus, one or more metrics associated with the health behavior goal(s) can be monitored for changes. As such, the application can provide recommendations to the user relating to the changes.

### SUMMARY

Aspects and advantages of embodiments of the present disclosure will be set forth in part in the following description, or can be learned from the description, or can be learned through practice of the embodiments.

In an aspect, the present disclosure is directed to a computing device having one or more processors and one or more computer-readable media that collectively store instructions that, when executed by the one or more processors, cause the computing device to perform operations. The operations include monitoring one or more metrics of physiological data of a user based at least in part on a health behavior goal of the user, the one or more metrics being associated with the health behavior goal, tracking adherence to the health behavior goal by the user over a defined time, detecting a statistical change to at least one metric value of the one or more metrics based at least in part on adherence to the health behavior goal by the user over the defined time, and performing one or more operations based at least in part on detecting the statistical change.

In another aspect, the present disclosure is directed to a computer-implemented method that includes monitoring one or more metrics of physiological data of a user based at least in part on receipt of input data indicative of a health behavior goal selected or defined by the user, the one or more metrics being associated with the health behavior goal, tracking adherence to the health behavior goal by the user over a defined time, detecting a statistical change to at least one metric value of the one or more metrics based at least in part on adherence to the health behavior goal by the user over the defined time, and performing one or more operations based at least in part on detecting the statistical change.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIGS. 1, 2, and 3 each illustrate a perspective view of an example, non-limiting wearable device according to one or more example embodiments of the present disclosure.

FIG. 4 illustrates a block diagram of an example, non-limiting device according to one or more example embodiments of the present disclosure.

FIG. 5 illustrates a diagram of an example, non-limiting user assessment management system according to one or more example embodiments of the present disclosure.

FIG. 6 illustrates a diagram of an example, non-limiting server system according to one or more example embodiments of the present disclosure.

FIGS. 7A-7D illustrate an example, non-limiting of a quantified-self program that can be implemented on a wearable device, an external computing device, and/or server system according to one or more example embodiments of the present disclosure, particularly illustrating initial explanation screens of the quantified-self program.

FIGS. 8A-8D illustrate an example, non-limiting of a quantified-self program that can be implemented on a wearable device, an external computing device, and/or server system according to one or more example embodiments of the present disclosure, particularly illustrating health behavior goal selection screens of the quantified-self program.

FIG. 9 illustrates an example, non-limiting of a quantified-self program that can be implemented on a wearable device, an external computing device, and/or server system according to one or more example embodiments of the present disclosure, particularly illustrating a goal achievability screen of the quantified-self program.

FIGS. 10A-10C illustrate an example, non-limiting of a quantified-self program that can be implemented on a wearable device, an external computing device, and/or server system according to one or more example embodiments of the present disclosure, particularly illustrating subjective question screens that can be displayed to a user of the quantified-self program.

FIG. 11 illustrates an example, non-limiting of a quantified-self program that can be implemented on a wearable device, an external computing device, and/or server system according to one or more example embodiments of the present disclosure, particularly illustrating a metric display screen of the quantified-self program.

FIG. 12 illustrates an example, non-limiting of a quantified-self program that can be implemented on a wearable device, an external computing device, and/or server system according to one or more example embodiments of the present disclosure, particularly illustrating a daily metric summary screen of the quantified-self program.

FIGS. 13A-13B illustrate an example, non-limiting of a quantified-self program that can be implemented on a wearable device, an external computing device, and/or server system according to one or more example embodiments of the present disclosure, particularly illustrating detailed metric information screens that can be displayed to a user of the quantified-self program.

FIG. 14 illustrates an example, non-limiting of a quantified-self program that can be implemented on a wearable device, an external computing device, and/or server system according to one or more example embodiments of the present disclosure, particularly illustrating an intelligent notification that can be displayed by the quantified-self program.

FIG. 15 illustrates an example, non-limiting of a flow diagram of a computer-implemented method according to one or more example embodiments of the present disclosure.

Repeated use of reference characters and/or numerals in the present specification and/or figures is intended to represent the same or analogous features, elements, or operations of the present disclosure. Repeated description of reference characters and/or numerals that are repeated in the present specification is omitted for brevity.

### DETAILED DESCRIPTION

### Overview

As referred to herein, the terms “includes” and “including” are intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling, etc.), mechanical coupling, operative coupling, optical coupling, and/or physical coupling.

As referenced herein, the term “system” can refer to hardware (e.g., application specific hardware), computer logic that executes on a general-purpose processor (e.g., a central processing unit (CPU)), and/or some combination thereof.

In some embodiments, a “system” described herein can be implemented in hardware, application specific circuits, firmware, and/or software controlling a general-purpose processor. In some embodiments, a “system” described herein can be implemented as program code files stored on a storage device, loaded into a memory, and executed by a processor, and/or can be provided from computer program products, for example, computer-executable instructions that are stored in a tangible computer-readable storage medium (e.g., random-access memory (RAM), hard disk, optical media, magnetic media).

As mentioned, individuals are unique and their motivational and adherence patterns in striving for a behavior goal can differ significantly. Health-related changes in response to a behavior change can also vary between people. The idea of self-experimentation is not new but the technology that enables it is relatively recent and advances in sensors and wearable technologies have made it increasingly possible for individuals to collect data about themselves with the goal of self-knowledge through personal data. However, gaining self-knowledge can be more challenging than only a simple task of data collection.

For example, the human body can produce biological signals that are non-stationary and stochastic in nature. Hence, it is natural for sensor-measured signals to fluctuate. These natural variations are not limited to biological signals. Rather, human behavior can also vary over time and even day-to-day due to personal or environmental factors as well as seasonal effects. Examples of these biological signals and health-related behaviors (health behaviors) may be, for example, daily resting heart rate, sleep duration, step counts, and bedtime.

Due to these natural variations, meaningful changes in biological signals or behaviors might not be easily recognizable. In the context of self-experimentation, for example, individuals might not recognize changes when the values are small relative to variability of corresponding signals.

Therefore, providing context to the change in measured signals can help individuals to understand their changes in response to a specific behavior change and the relationship between the two. Moreover, understanding daily variations of the biological signals and corresponding distributions among populations of the same demographics can provide valuable context into interpreting biological signal values measured by wearables and changes associated with a behavior.

Motivated by these gaps in understanding personal data, the inventors of the present disclosure developed a quantified-self program for a computer application that is configured to reduce the burden of self-experimentation by enabling daily behavior and health change tracking. Thus, the computer application of the present disclosure is configured to support individuals in understanding the relationship between their behavior and changes in their metrics by applying self-experimentation. In particular, the computer application of the present disclosure enables participants to establish a baseline for their health metrics, select a health-related behavior goal and track their daily adherence in addition to rigorous insights, contextual information, and data visualizations to help participants understand their personalized response to the change.

According to example embodiments of the present disclosure, a computing device (e.g., a server, a client computing device, a computer, a laptop, a tablet, a smartphone, a physiological monitoring device, a wearable computing device, a wearable physiological monitoring device (e.g., a wrist-worn device, a chest strap device)) can monitor one or more metrics of physiological data of a user based at least in part on a health behavior goal of the user. The metric(s) may be associated with the health behavior goal. In one or more embodiments, the physiological data can be captured by one or more sensors (e.g., physiological sensors) of the computing device. As such, the computing device can obtain such physiological data from such a wearable physiological monitoring device by using, for instance, a network (e.g., the Internet) as described in example embodiments of the present disclosure. In at least one embodiment, such physiological data can constitute, include, and/or otherwise be associated with, for instance: heart rate (HR) data, motion data (e.g., accelerometer data), respiration rate data, blood pressure data, blood oxygenation level data, body temperature data, data associated with (e.g., indicative or descriptive of) the user's deoxyribonucleic acid (DNA), blood glucose data, electrodermal activity (EDA) data, stress related data, and/or other physiological data that can be captured by, for instance, a wearable physiological monitoring device (e.g., a wrist-worn device, a chest strap device) according to example embodiments described herein and/or another physiological monitoring device.

Further, the computing device can track adherence to the health behavior goal by the user over a defined time. Moreover, the computing device may detect a statistical change to at least one metric value of the metric(s) based at least in part on adherence to the health behavior goal by the user over the defined time and when possible attribute the change in health metric to change in behavior while controlling for seasonality and other behaviors tracked passively. In addition, the computing device may perform one or more operations based at least in part on detecting the statistical change.

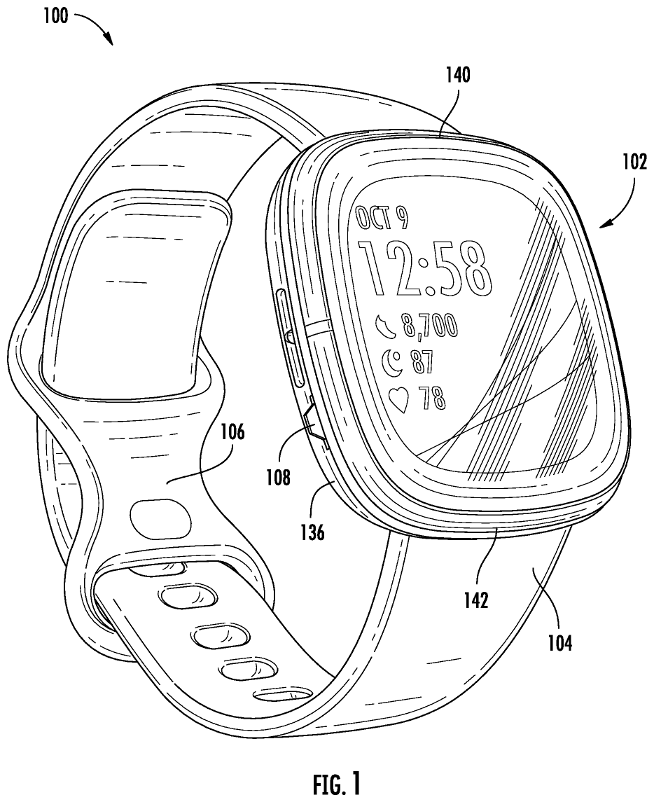

In one or more embodiments, the computing device described above and below according to example embodiments of the present disclosure can constitute, include, be coupled to, and/or otherwise be associated with one or more computing devices and/or computing systems described below and illustrated in the example embodiments depicted in FIGS. 1-6. For example, in at least one embodiment, the computing device described above and below according to example embodiments of the present disclosure can constitute, include, be coupled to, and/or otherwise be associated with wearable device, external computing device, and/or server system.

In the above embodiment, wearable device, external computing device, and/or server system can individually and/or collectively perform the physiological monitoring and/or the health, wellness, and/or well-being assessment operations described herein (e.g., the physical, mental, emotional, behavioral, and/or sleep quality assessment operations) in accordance with one or more embodiments of the present disclosure. In this embodiment, based at least in part on (e.g., in response to) performing such assessment operations, wearable device, external computing device, and/or server system can further perform, individually and/or collectively, one or more operations described herein that can facilitate alteration (e.g., improvement) of a user's health quality in accordance with one or more embodiments of the present disclosure.

Example aspects of the present disclosure provide several technical effects, benefits, and/or improvements in computing technology.

### Example Devices and Systems

FIGS. 1, 2, and 3 each illustrate a perspective view of an example, non-limiting wearable device 100 according to one or more example embodiments of the present disclosure. In example embodiments described herein, wearable device 100 can constitute and/or include a wearable computing device. For instance, in these or other example embodiments, wearable device 100 can constitute and/or include a wearable computing device such as, for example, a wearable physiological monitoring device that can be worn by a user (also referred to herein as a “wearer”) and/or capture one or more types of physiological data of the user (e.g., heart rate (HR) data, motion data (e.g., accelerometer data), body temperature data, respiration rate data, blood pressure data, blood oxygenation level data, deoxyribonucleic acid (DNA) data, electrodermal activity (EDA) data, stress related data).

Wearable device 100 according to example embodiments of the present disclosure can include a display 102, an attachment component 104, a securement component 106, and a button 108 that can be located on a side of wearable device 100. In at least one embodiment, two sides of display 102 can be coupled (e.g., mechanically, operatively) to attachment component 104. In some embodiments, securement component 106 can be located on, coupled to (e.g., mechanically, operatively), and/or integrated with attachment component 104. In these or other embodiments, securement component 106 can be positioned opposite display 102 on an opposing end of attachment component 104. In some embodiments, button 108 can be located on a side of wearable device 100, underneath display 102.

Display 102 according to example embodiments described herein can constitute and/or include any type of electronic display or screen known in the art. For example, in some embodiments, display 102 can constitute and/or include a liquid crystal display (LCD) or organic light emitting diode (OLED) display such as, for instance, a transmissive LCD display or a transmissive OLED display. Display 102 according to example embodiments can be configured to provide brightness, contrast, and/or color saturation features according to display settings that can be maintained by control circuitry and/or other internal components and/or circuitry of wearable device 100. In some embodiments, display 102 can constitute and/or include a touchscreen such as, for instance, a capacitive touchscreen. For example, in these embodiments, display 102 can constitute and/or include a surface capacitive touchscreen or a projective capacitive touch screen that can be configured to respond to contact with electrical charge-holding members or tools, such as a human finger.

In some embodiments, display 102 can be configured to provide (e.g., render) a variety of information such as, for example, the time, the date, body signals (e.g., physiological data of a user wearing wearable device 100), readings based upon user input, and/or other information. In one embodiment, such body signals can include, but are not limited to, heart rate data (e.g., heart beats per minute), motion data (e.g., movement data, accelerometer data), blood pressure data, body temperature data, respiration rate data, blood oxygenation level data, deoxyribonucleic acid (DNA) data, electrodermal activity (EDA) data, stress related data and/or any other body signal that one of ordinary skill in the art would understand that can be measured by a wearable device such as, for instance, wearable device 100. In some embodiments, the readings based upon user input can include, but are not limited to, the number of steps a user has taken, the distance traveled by the user, the sleep schedule of the user, travel routes of the user, elevation climbed by the user, and/or any other metric that one of ordinary skill in the art would understand that can be input by a user into a wearable device such as, for instance, wearable device 100.

In at least one embodiment of the present disclosure, the above-described body signals and/or readings based upon user input can be used to calculate further analytics to provide a user with data such as, for instance, a fitness score, a sleep quality score, a number of calories burned by the user, and/or other data. In some embodiments, wearable device 100 can take in (e.g., capture, collect, receive, measure) outside data irrespective of the user such as, for example: an ambient temperature of an environment surrounding and/or external to wearable device 100; an amount of sun exposure wearable device 100 is subjected to; an atmospheric pressure of the environment surrounding and/or external to wearable device 100; an air quality of the environment surrounding and/or external to wearable device 100; the location of wearable device 100 based on, for instance, a global positioning system (GPS); and/or other outside factors that one of ordinary skill in the art would understand a wearable device such as, for instance, wearable device 100 can take in (e.g., capture, collect, receive, measure).

Attachment component 104 according to example embodiments described herein can be used to attach (e.g., affix, fasten) wearable device 100 to a user of wearable device 100. In some embodiments, attachment component 104 can take the form of, for example, a strap, an elastic band, a rope, and/or any other form of attachment one of ordinary skill in the art would understand can be used to attach a wearable device such as, for instance, wearable device 100 to a user.

Securement component 106 according to example embodiments of the present disclosure can facilitate attachment of attachment component 104 upon a user of wearable device 100. In some embodiments, securement component 106 can include, but is not limited to, a pin and hole locking mechanism (e.g., a buckle), a magnet system, a lock, a clip, and/or any other type of securement that one of ordinary skill would understand can be used to facilitate attachment of a wearable device such as, for instance, wearable device 100 to a user. In one embodiment, wearable device 100 does not include securement component 106. For example, in this or another embodiment, wearable device 100 can be secured to a user with a strap that can be tied around the user's wrist and/or another suitable appendage.

Button 108 according to example embodiments described herein can allow for a user to interact with wearable device 100 and/or allow for the user to provide a form of input into wearable device 100. In the example embodiment depicted in FIGS. 1, 2, and 3, one button 108 is shown on wearable device 100. However, it should be appreciated that wearable device 100 is not so limiting. For example, in some embodiments, wearable device 100 can include any number of buttons that allow a user to further interact with wearable device 100 and/or to provide alternative inputs. In at least one embodiment, wearable device 100 does not include button 108. For instance, as described above, in example embodiments, wearable device 100 can include a screen such as, for example, a touch screen that can receive inputs through (e.g., by way of) the touch of the user. In additional or alternative embodiments, wearable device 100 can include a microphone that can receive inputs through (e.g., by way of) voice commands of a user.

By way of example, in further embodiments, wearable device 100 can generate, configure, and/or render an interactive user interface such as, for instance, an interactive button wheel on a touch screen coupled to the wearable device 100. In this and/or another embodiment, wearable device 100 can generate, configure, and/or render the interactive button wheel such that it has multiple interactive buttons (e.g., 5, 10, 15, 20, etc.). In this and/or another embodiment, each of such interactive buttons can be configured by wearable device 100 such that they can receive input from the user 10 by way of a touch (e.g., fingertip touch) by the user 10 to indicate a selection by the user 10.

In some embodiments, wearable device 100 can constitute a portable computing device that can be designed so that it can be inserted into a wearable case (e.g., as illustrated in the example embodiments depicted in FIGS. 1, 2, and 3). In some embodiments, wearable device 100 can constitute a portable computing device that can be designed so that it can be inserted into one or more of multiple different wearable cases (e.g., a wristband case, a belt-clip case, a pendant case, a case configured to be attached to a piece of exercise equipment such as a bicycle). Wearable device 100 according to embodiments described herein can be formed into one or more shapes and/or sizes to allow for coupling to (e.g., secured to, worn, borne by) the body or clothing of a user. In some embodiments, wearable device 100 can constitute a portable computing device that can be designed to be worn in limited manners such as, for instance, a computing device that is integrated into a wristband in a non-removable manner and/or can be intended to be worn specifically on a person's wrist (or perhaps ankle).

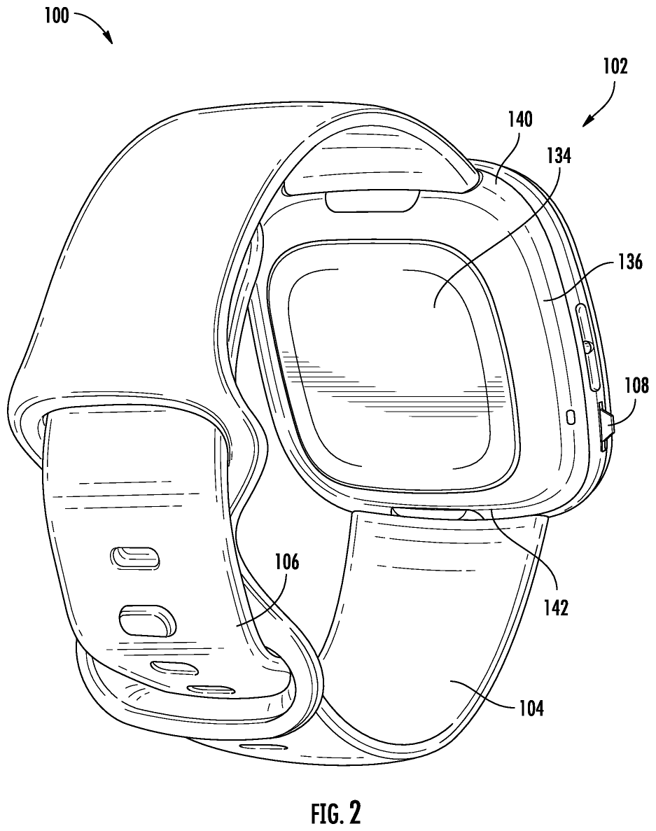

In another embodiment, wearable device 100 according to example embodiments of the present disclosure can include one or more physiological and/or environmental sensors (e.g., internal physiological sensor(s) 143, external physiological sensor(s) 145, and/or environmental sensor(s) 155 described below with reference to FIG. 4) that can be configured to collect physiological and/or environmental data in accordance with various embodiments disclosed herein. In some embodiments, wearable device 100 can be configured to analyze and/or interpret collected physiological and/or environmental data to perform one or more health, wellness, and/or well-being assessments (e.g., physical, mental, emotional, behavioral, and/or sleep quality assessment(s)) of a user (e.g., a wearer) of wearable device 100 according to one or more embodiments described herein. In additional and/or alternative embodiments, wearable device 100 can be configured to communicate with another computing device or server that can perform such one or more health, wellness, and/or well-being assessments (e.g., physical, mental, emotional, behavioral, and/or sleep quality assessment(s)) of a user (e.g., a wearer) of wearable device 100 according to one or more embodiments described herein.

Wearable device 100 in accordance with one or more example embodiments of the present disclosure can include one or more physiological and/or environmental components and/or modules that can be designed to determine one or more physiological and/or environmental metrics associated with a user (e.g., a wearer) of wearable device 100. In at least one embodiment, such physiological and/or environmental component(s) and/or module(s) can constitute and/or include one or more physiological and/or environmental sensors. For instance, although not depicted in the example embodiments illustrated in FIGS. 1, 2, and 3, in some embodiments, wearable device 100 can include one or more physiological and/or environmental sensors such as, for example, an accelerometer, a heart rate sensor (e.g., photoplethysmography (PPG) sensor), an electrodermal activity (EDA) sensor, a body temperature sensor, an environment temperature sensor, and/or another physiological and/or environmental sensor. In these or other embodiments, such physiological and/or environmental sensor(s) can be disposed on, coupled to, and/or otherwise be associated with an underside and/or a backside (e.g., back 134) of wearable device 100.

In some embodiments, the above-described physiological and/or environmental sensor(s) can be disposed on, coupled to, and/or otherwise be associated with wearable device 100 such that the sensor(s) can be in contact with or substantially in contact with human skin when wearable device 100 is worn by a user. For example, in embodiments where wearable device 100 can be worn on a user's wrist, the physiological and/or environmental sensor(s) can be disposed on, coupled to, and/or otherwise be associated with back 134 that can be substantially opposite display 102 and touching an arm of the user. In one embodiment, the above-described physiological and/or environmental sensor(s) can be disposed on, coupled to, and/or otherwise be associated with an interior or skin-side of wearable device 100 (e.g., a side of wearable device 100 that contacts, touches, and/or faces the skin of the user such as, for instance, back 134 and/or bottom 142). In another embodiment, the physiological and/or environmental sensors can be disposed on one or more sides of wearable device 100, including the skin-side (e.g., back 134, bottom 142) and one or more sides (e.g., first side 136, second side 138, top 140, display 102) of wearable device 100 that face and/or are exposed to the ambient environment (e.g., the external environment surrounding wearable device 100).

Referring now to FIG. 4, a block diagram of the above-described example, non-limiting wearable device 100 according to one or more example embodiments of the present disclosure is illustrated. That is, for instance, FIG. 4 illustrates a block diagram of one or more internal and/or external components of the above-described example, non-limiting wearable device 100 according to one or more example embodiments of the present disclosure.

As described above with reference to the example embodiments depicted in FIGS. 1, 2, and 3, wearable device 100 can constitute and/or include a wearable computing device such as, for instance, a wearable physiological monitoring device. For example, in the example embodiment depicted in FIG. 4, wearable device 100 can constitute and/or include a wearable physiological monitoring device that can be worn by a user 10 (also referred to herein as a “wearer” or “wearer 10”) and/or can be configured to gather data regarding activities performed by user 10 and/or data regarding user's 10 physiological (e.g., physical), mental, and/or emotional state (e.g., including sleep quality). In this or another embodiment, such data can include data representative of the ambient environment around user 10 or user's 10 interaction with the environment. For example, in some embodiments, the data can constitute and/or include motion data regarding user's 10 movements, ambient light, ambient noise, air quality, and/or physiological data obtained by measuring various physiological characteristics of user 10 (e.g., heart rate, respiratory data, body temperature, blood oxygen levels, perspiration levels, movement data).

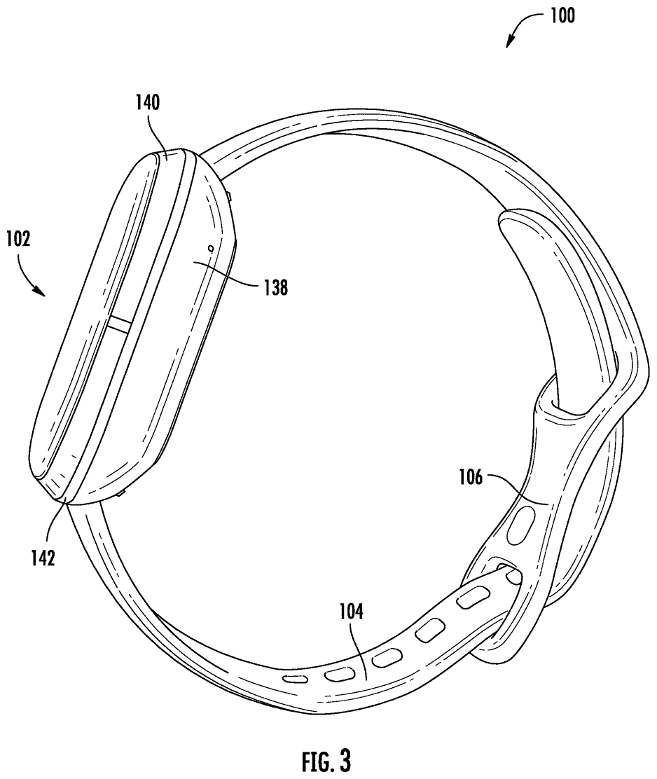

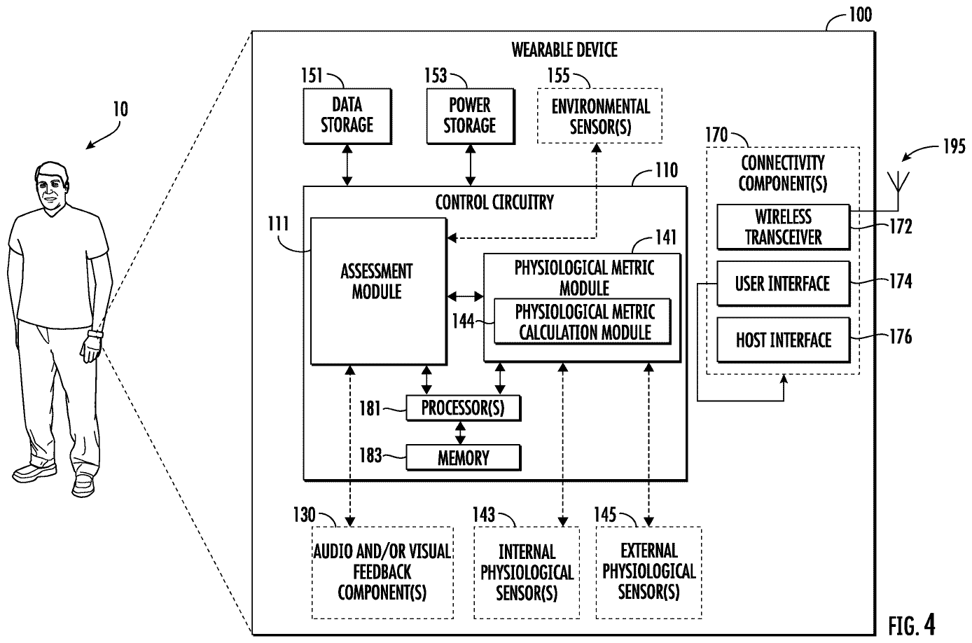

Although certain embodiments are disclosed herein in the context of wearable physiological monitoring devices, it should be appreciated that the present disclosure is not so limiting. For example, it should be understood that the physiological monitoring and the health, wellness, and/or well-being assessment principles and features disclosed herein (e.g., physical, mental, emotional, behavioral, and/or sleep quality assessment) can be applicable with respect to and/or implemented using any suitable or desirable type of computing device or combination of computing devices, whether wearable or not. For instance, the physiological monitoring and the health, wellness, and/or well-being assessment principles and features disclosed herein (e.g., physical, mental, emotional, behavioral, and/or sleep quality assessment) in accordance with one or more embodiments can by performed and/or implemented using any suitable or desirable type of computing device or combination of computing devices such as, for example, a client computing device, a laptop, a tablet, a server (e.g., server system 512 described below and depicted in FIG. 6), a wearable computing device (e.g., wearable device 100), a smartphone (e.g., an external computing device 504 described below and depicted in FIG. 5), and/or another computing device, whether wearable or not.

As illustrated in FIG. 4, wearable device 100 according to example embodiments of the present disclosure can include one or more audio and/or visual feedback components 130 such as, for instance, electronic touchscreen display units, light-emitting diode (LED) display units, audio speakers, light-emitting diode (LED) lights, buzzers, and/or another type of audio and/or visual feedback module. In certain embodiments, one or more audio and/or visual feedback modules 130 can be located on and/or otherwise associated with a front side of wearable device 100 and/or display 102. For example, in wearable embodiments of wearable device 100, an electronic display such as, for instance, display 102 can be configured to be externally presented to user 10 viewing wearable device 100.

Wearable device 100 according to example embodiments of the present disclosure can include control circuitry 110. Although certain modules and/or components are illustrated as part of control circuitry 110 in the diagram of FIG. 4, it should be understood that control circuitry 110 associated with wearable device 100 and/or other components or devices in accordance with example embodiments of the present disclosure can include additional components and/or circuitry such as, for instance, one or more additional components of the illustrated components depicted in FIG. 4. Furthermore, in certain embodiments, one or more of the illustrated components of control circuitry 110 can be omitted and/or different than that shown in FIG. 4 and described in association therewith.

The term “control circuitry” is used herein according to its broad and/ordinary meaning and can include any combination of software and/or hardware elements, devices, and/or features that can be implemented in connection with operation of wearable device 100. Furthermore, the term “control circuitry” can be used substantially interchangeably in certain contexts herein with one or more of the terms “controller,” “integrated circuit,” “IC,” “application-specific integrated circuit,” “ASIC,” “controller chip,” or the like.

Control circuitry 110 according to example embodiments of the present disclosure can constitute and/or include one or more processors, data storage devices, and/or electrical connections. In one embodiment, control circuitry 110 can be implemented on a system on a chip (SoC), however, those skilled in the art will recognize that other hardware and/or firmware implementations are possible.

In one or more embodiments of the present disclosure, control circuitry 110 can constitute and/or include one or more processors 181 that can be configured to execute computer-readable instructions that, when executed, cause wearable device 100 to perform one or more operations. In at least one embodiment, control circuitry 110 can constitute and/or include processor(s) 181 that can be configured to execute operational code (e.g., instructions, processing threads, software) for wearable device 100 such as, for instance, firmware or the like. Processor(s) 181 according to example embodiments described herein can each be a processing device. For instance, in the example embodiment depicted in FIG. 4, processor(s) 181 can each be a central processing unit (CPU), microprocessor, microcontroller, integrated circuit (e.g., an application-specific integrated circuit (ASIC)), and/or another type of processing device. In this or another example embodiment, processor(s) 181 can be coupled to (e.g., electrically, communicatively, physically, operatively) to one or more components of control circuitry 110 and/or wearable device 100 such that processor(s) 181 can facilitate one or more operations in accordance with one or more example embodiments described herein.

In at least one embodiment of the present disclosure, the above-described computer-readable instructions and/or operational code that can be executed by processor(s) 181 can be stored in one or more data storage devices of wearable device 100. In the example embodiment depicted in FIG. 4, such computer-readable instructions and/or operational code can be stored in memory 183 of wearable device 100. In this or another example embodiment, memory 183 can be coupled to (e.g., electrically, communicatively, physically, operatively) to one or more components of control circuitry 110 and/or wearable device 100 such that memory 183 can facilitate one or more operations in accordance with one or more example embodiments described herein.

Memory 183 according to example embodiments described herein can store computer-readable and/or computer executable entities (e.g., data, information, applications, models, algorithms) that can be created, modified, accessed, read, retrieved, and/or executed by each of processor(s) 181. In some embodiments, memory 183 can constitute, include, be coupled to (e.g., operatively), and/or otherwise be associated with a computing system and/or media such as, for example, one or more computer-readable media, volatile memory, non-volatile memory, random-access memory (RAM), read only memory (ROM), hard drives, flash drives, and/or other memory devices. In these or other embodiments, such one or more computer-readable media can include, constitute, be coupled to (e.g., operatively), and/or otherwise be associated with one or more non-transitory computer-readable media. Although not depicted in the example embodiment illustrated in FIG. 4, in some embodiments, memory 183 can include (e.g., store) an assessment module 111, physiological metric module 141, physiological metric calculation module 144, and/or other modules and/or data that can be used to facilitate one or more operations described herein.

Control circuitry 110 according to example embodiments of the present disclosure can constitute and/or include assessment module 111. Assessment module 111 according to example embodiments of the present disclosure can constitute and/or include one or more hardware and/or software components and/or features that can be configured to perform one or more assessments of user 10 in accordance with one or more embodiments described herein. For example, in some embodiments, assessment module 111 can constitute and/or include one or more hardware and/or software components and/or features that can be configured to perform one or more health, wellness, and/or well-being assessments (e.g., physical, mental, emotional, behavioral, and/or sleep quality assessment(s)) of user 10 according to one or more embodiments described herein. In some embodiments, to perform such assessment(s), assessment module 111 can use inputs from one or more environmental sensors 155 (e.g., ambient light sensor) and/or information from physiological metric module 141.

In one embodiment, assessment module 111 can constitute and/or include a machine learning (ML) and/or artificial intelligence (AI) model (e.g., a function, algorithm, process). In one embodiment, wearable device 100 can train such ML and/or AI model(s) as described herein. In one embodiment, wearable device 100 can implement (e.g., execute, run) such ML and/or AI model(s) using physiological data of user 10 that can be accumulated by assessment module 111 such as, for instance, the values of one or more physiological metrics (e.g., user's 10 heart rate, motion, temperature, respiration, perspiration, electrodermal activity (EDA)) that can be determined by physiological metric calculation module 144 of physiological metric module 141.

In certain embodiments, physiological metric module 141 and/or physiological metric calculation module 144 can be communicatively coupled with one or more internal physiological sensors 143 that can be embedded and/or integrated in wearable device 100. In certain embodiments, physiological metric module 141 and/or physiological metric calculation module 144 can be optionally in communication with one or more external physiological sensors 145 not embedded and/or integrated in wearable device 100 (e.g., an electrode or sensor integrated in another electronic device). In some embodiments, examples of internal physiological sensors 143 and/or external physiological sensors 145 can constitute and/or include, but are not limited to, one or more sensors that can measure (e.g., capture, collect, receive) physiological data of user 10 such as, for instance, body temperature, heart rate, blood oxygen level, movement, respiration, perspiration, electrodermal activity (EDA), stress data, and/or other physiological data of user 10.

In the example embodiment depicted in FIG. 4, wearable device 100 can include one or more data storage components 151 (denoted as “data storage 151” in FIG. 4). Data storage component(s) 151 according to example embodiments can constitute and/or include any suitable or desirable type of data storage such as, for instance, solid-state memory, which can be volatile or non-volatile. In some embodiments, such solid-state memory of wearable device 100 can constitute and/or include any of a wide variety of technologies such as, for instance, flash integrated circuits, phase change (PC) memory, phase change (PC) random-access memory (RAM), programmable metallization cell RAM (PMC-RAM or PMCm), ovonic unified memory (OUM), resistance RAM (RRAM), NAND memory, NOR memory, EEPROM, ferroelectric memory (FeRAM), MRAM, or other discrete NVM (non-volatile solid-state memory) chips. In some embodiments, data storage component(s) 151 can be used to store system data, such as operating system data and/or system configurations or parameters. In some embodiments, wearable device 100 can include data storage utilized as a buffer and/or cache memory for operational use by control circuitry 110.

Data storage component(s) 151 according to example embodiments can include various sub-modules that can be implemented to facilitate the physiological monitoring and the health, wellness, and/or well-being assessment principles and features disclosed herein (e.g., physical, mental, emotional, behavioral, and/or sleep quality assessment) in accordance with one or more embodiments. For example, in at least one embodiment, data storage 151 can include one or more sub-modules that can include, but not limited to: an information collection module (e.g., physiological metric module 141, physiological metric calculation module 144) that can manage the collection of physiological and/or environmental data relevant to any health, wellness, and/or well-being assessment described herein (e.g., physical, mental, emotional, behavioral, and/or sleep quality assessment(s)); a heart rate determination module that can determine values and/or patterns of one or more types of heart rates of user 10; assessment module 111; a sleep detection module that can detect an attempt or onset of sleep by the user 10; a presentation module that can manage presentation of information to user 10 that can be associated with any health, wellness, and/or well-being assessment described herein (e.g., physical, mental, emotional, behavioral, and/or sleep quality assessment(s)); a feedback management module for collecting and interpreting any input data and/or feedback received from user 10 (e.g., information associated with user's 10 physical, mental, emotional, behavioral, and/or sleep quality state); and/or another sub-module.

Wearable device 100 according to example embodiments can further include a power storage module 153 (denoted as “power storage 153”), which can constitute and/or include a rechargeable battery, one or more capacitors, or other charge-holding device(s). In some embodiments, the power stored by power storage module 153 can be utilized by control circuitry 110 for operation of wearable device 100, such as for powering display 102. In some embodiments, power storage module 153 can receive power over a host interface of wearable device 100 (e.g., via one or more host interface circuitry and/or components 176 (denoted as “host interface 176” in FIG. 4)) and/or through other means.

Wearable device 100 according to example embodiments can further include one or more environmental sensors 155. In at least one embodiment, examples of such environmental sensors 155 can include, but are not limited to, sensors that can determine and/or measure, for instance, ambient light, external (non-body) temperature, altitude, device location (e.g., global-positioning system (GPS)), and/or another environmental data.

Wearable device 100 according to example embodiments can further include one or more connectivity components 170, which can include, for example, a wireless transceiver 172. Wireless transceiver 172 according to example embodiments can be communicatively coupled to one or more antenna devices 195, which can be configured to wirelessly transmit and/or receive data and/or power signals to and/or from wearable device 100 using, but not limited to, peer-to-peer, WLAN, and/or cellular communications. For example, wireless transceiver 172 can be utilized to communicate data and/or power between wearable device 100 and an external computing device (not illustrated in FIG. 4) such as, for instance, an external client computing device (e.g., a smartphone, tablet, computer) and/or an external host system (e.g., a server), which can be configured to interface with wearable device 100. In certain embodiments, wearable device 100 can include one or more host interface circuitry and/or components 176 (denoted as “host interface 176” in FIG. 4) such as, for instance, wired interface components that can communicatively couple wearable device 100 with the above-described external computing device (e.g., a smartphone, table, computer, server) to receive data and/or power therefrom and/or transmit data thereto.

Connectivity component(s) 170 according to example embodiments can further include one or more user interface components 174 (denoted as “user interface 174” in FIG. 4) that can be used by wearable device 100 to receive input data from user 10 and/or provide output data to user 10. In some embodiments, user interface component(s) 174 can be coupled to (e.g., operatively, communicatively) and/or otherwise be associated with audio and/or visual feedback component(s) 130. For instance, in these embodiments, display 102 of wearable device 100 can constitute and/or include a touchscreen display that can be configured to provide (e.g., render) output data to user 10 and/or to use audio and/or visual feedback component(s) 130 to receive user input through user contact with the touchscreen display. In some embodiments, user interface component(s) 174 can further constitute and/or include one or more buttons or other input components or features.

Connectivity component(s) 170 according to example embodiments can further include host interface circuitry and/or component(s) 176, which can be, for example, an interface that can be used by wearable device 100 to communicate with the above-described external computing device (e.g., a smartphone, table, computer, server) over a wired or wireless connection. Host interface circuitry and/or component(s) 176 according to example embodiments can utilize and/or otherwise be associated with any suitable or desirable communication protocol and/or physical connector such as, for instance, universal serial bus (USB), micro-USB, Wi-Fi, Bluetooth, Fire Wire, PCIe, or the like. For wireless connections, host interface circuitry and/or component(s) 176 according to example embodiments can be incorporated with wireless transceiver 172.

Although certain functional modules and components are illustrated and described herein, it should be understood that authentication management functionality in accordance with the present disclosure can be implemented using a number of different approaches. For example, in some embodiments, control circuitry 110 can constitute and/or include one or more processors (e.g., processor(s) 181) that can be controlled by computer-executable instructions that can be stored in a memory (e.g., memory 183, data storage component(s) 151) so as to provide functionality such as is described herein. In other embodiments, such functionality can be provided in the form of one or more specially designed electrical circuits. In some embodiments, such functionality can be provided by one or more processors (e.g., processor(s) 181) that can be controlled by computer-executable instructions that can be stored in a memory (e.g., memory 183, data storage component(s) 151) that can be coupled to (e.g., communicatively, operatively, electrically) one or more specially designed electrical circuits. Various examples of hardware that can be used to implement the concepts outlined herein can include, but are not limited to, application specific integrated circuits (ASICs), field-programmable gate arrays (FPGAs), and general-purpose microprocessors that can be coupled with memory that stores executable instructions for controlling the general-purpose microprocessors.

FIG. 5 illustrates a diagram of an example, non-limiting user assessment management system 500 according to one or more example embodiments of the present disclosure. User assessment management system 500 depicted in FIG. 5 illustrates an example, non-limiting networked relationship between wearable device 100 and external computing device 504 in accordance with one or more embodiments.

With reference to the example embodiment described above and depicted in FIG. 4, wearable device 100 according to example embodiments of the present disclosure can perform one or more health, wellness, and/or well-being assessments (e.g., physical, mental, emotional, behavioral, and/or sleep quality assessment(s)) of user 10 and/or perform operation(s) to facilitate alteration (e.g., improvement) of user's 10 health, wellness, and/or well-being based on such assessment(s). As such, in certain embodiments described in the present disclosure, wearable device 100 can be capable of and/or configured to collect physiological sensor readings of user 10 and/or perform such assessment(s) and/or operation(s) using such readings.

However, in additional and/or alternative embodiments, wearable device 100 and/or another electronic and/or computing device that can be used to detect physiological information of user 10, can be in communication with external computing device 504. In these and/or other embodiments, external computing device 504 can be configured to use such physiological information of user 10 to perform such one or more health, wellness, and/or well-being assessments (e.g., physical, mental, emotional, behavioral, and/or sleep quality assessment(s)) of user 10 according to one or more embodiments described herein. In these and/or other embodiments, based at least in part on (e.g., in response to) performing such assessment(s), external computing device 504 can perform one or more operations described herein to facilitate alteration (e.g., improvement) of user's 10 health, wellness, and/or well-being (e.g., physical, mental, emotional, behavioral, and/or sleep quality).

Wearable device 100 according to example embodiments can be configured to collect one or more types of physiological and/or environmental data using embedded sensors and/or external devices, as described throughout the present disclosure, and communicate or relay such information over one or more networks 506 to other devices. This includes, in some embodiments, relaying information to devices capable of serving as Internet-accessible data sources, thus permitting the collected data to be viewed, for example, using a web browser or network-based application at, for instance, external computing device 504. For example, while user 10 is wearing wearable device 100, wearable device 100 can capture, calculate, and/or store environment data and/or user's 10 physiological data (e.g., heart rate, motion data, temperature, respiration, perspiration, EDA, stress data) using one or more environmental and/or physiological sensors. Wearable device 100 according to example embodiments can then transmit data representative of such environment data and/or user's 10 physiological data over network(s) 506 to an account on a web service, computer, mobile phone, and/or health station where the data can be stored, processed, and visualized by user 10 and/or another entity (e.g., a health care professional).

While wearable device 100 is shown in example embodiments of the present disclosure to have a display, it should be understood that, in some embodiments, wearable device 100 does not have any type of display unit. In some embodiments, wearable device 100 can have audio and/or visual feedback components such as, for instance, light-emitting diodes (LEDs), buzzers, speakers, and/or a display with limited functionality. Wearable device 100 according to example embodiments can be configured to be attached to user's 10 body or clothing. For example, in these or other embodiments, wearable device 100 can be configured as a wrist bracelet, watch, ring, electrode, finger-clip, toe-clip, chest-strap, ankle strap, and/or a device placed in a pocket. In additional or alternative embodiments, wearable device 100 can be embedded in something in contact with user 10 such as, for instance, clothing, a mat that can be positioned under user 10, a blanket, a pillow, and/or another accessory.

In one or more embodiments of the present disclosure, the communication between wearable device 100 and external computing device 504 can be facilitated by network(s) 506. In some embodiments, network(s) 506 can constitute and/or include, for instance, one or more of an ad hoc network, a peer-to-peer communication link, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the public switched telephone network (PSTN), a cellular telephone network, and/or any other type of network. In some embodiments, the communication between wearable device 100 and external computing device 504 can also be performed through a direct wired connection. In these or other embodiments, this direct-wired connection can be associated with any suitable or desirable communication protocol and/or physical connector such as, for instance, universal serial bus (USB), micro-USB, Wi-Fi, Bluetooth, FireWire, PCIe, or the like.

In example embodiments of the present disclosure, a variety of computing devices can be in communication with wearable device 100 to facilitate user's 10 health, wellness, and/or well-being assessment and/or alteration (e.g., improvement). Although external computing device 504 is depicted as a smartphone in the example embodiment illustrated in FIG. 5, it should be understood that the present disclosure is not so limiting. For instance, external computing device 504 according to example embodiments can constitute and/or include, for example, a smartphone with a display 508 as depicted in FIG. 5, a personal digital assistant (PDA), a mobile phone, a tablet, a personal computer, a laptop computer, a smart television, a video game console, a server, and/or another computing device that can be external to wearable device 100.

The networked relationship depicted in the example embodiment illustrated in FIG. 5 demonstrates how, in some embodiments, external computing device 504 can be implemented to perform one or more health, wellness, and/or well-being assessments (e.g., physical, mental, emotional, behavioral, and/or sleep quality assessment(s)) of user 10 and/or perform operation(s) to facilitate alteration (e.g., improvement) of user's 10 health, wellness, and/or well-being based on such assessment(s). For example, in one embodiment, user 10 can wear wearable device 100 that can be equipped as a bracelet with one or more physiological sensors but without a display. In this and/or another embodiment, while user 10 is wearing wearable device 100, wearable device 100 can capture, calculate, and/or store environment data and/or user's 10 physiological data (e.g., heart rate, motion data, temperature, respiration, perspiration, EDA, stress data) using one or more environmental and/or physiological sensors. Wearable device 100 according to example embodiments can then transmit data representative of such environment data and/or user's 10 physiological data over network(s) 506 to an account on a web service, computer, mobile phone, and/or health station where the data can be stored, processed, and visualized by user 10 and/or another entity (e.g., a health care professional). In some embodiments, wearable device 100 can periodically or continuously transmit such information to external computing device 504 over network(s) 506. In additional and/or alternative embodiments, wearable device 100 can store the above-described collected physiological and/or environmental data in a local memory and/or transmit this data to external computing device 504.

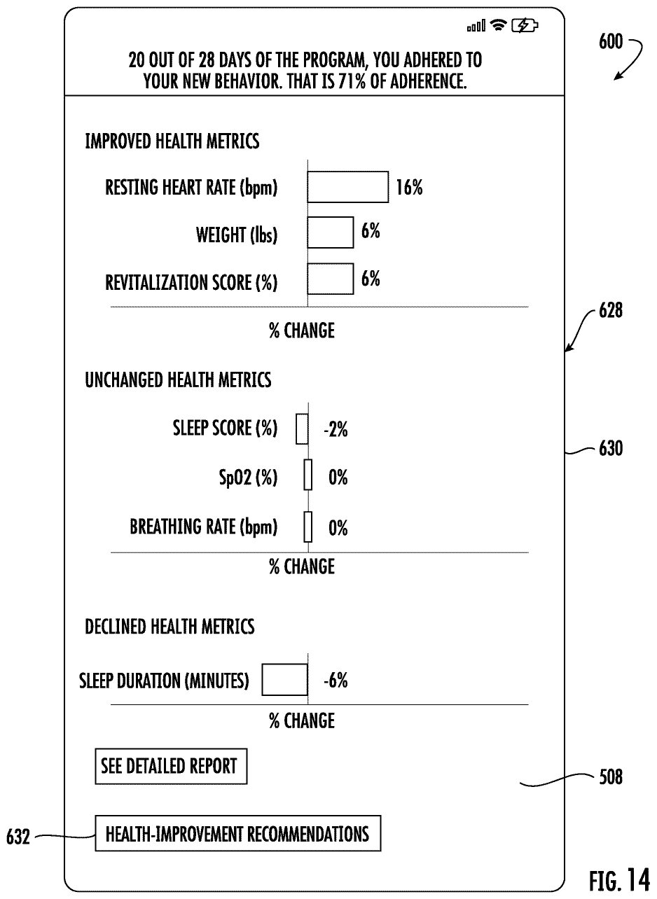

Accordingly, in an embodiment, external computing device 504 is configured to generate an intelligent notification 510 and provide the intelligent notification 510 to the user 10, e.g., via display 508 or a second computing device. In some embodiments, the intelligent notification 510 can include one or more health improvement recommendations 632 (see e.g., FIGS. 5 and 14), which will be described in more detail herein below.

Referring to FIGS. 5 and 6, user assessment management system 500 may further include a server system 512 in accordance with one or more embodiments. In the example embodiment, server system 512 can collect detected physiological and/or environmental sensor readings from wearable device 100. As another example, in the embodiment depicted in FIG. 5, wearable device 100 can transmit physiological data of user 10 to server system 512. In this embodiment, external computing device 504 can analyze the received data. In this embodiment, server system 512 can use the received physiological data to update a user profile for user 10 that can be stored in a database 514 (e.g., a log) of a memory 516 of server system 512.

In some embodiments, server system 512 can be implemented on one or more standalone data processing apparatuses or a distributed network of computers. In some embodiments, server system 512 can employ various virtual devices and/or services of third-party service providers (e.g., third-party cloud service providers) to provide the underlying computing resources and/or infrastructure resources of server system 512. In some embodiments, server system 512 can include, but is not limited to, a handheld computer, a tablet computer, a laptop computer, a desktop computer, or a combination of any two or more of these data processing devices or other data processing devices.

Server system 512 according to example embodiments can include one or more processors 518 (e.g., processing unit(s), denoted as “processor(s) 518” in FIG. 6) such as, for instance, one or more CPUs. In these or other embodiments, server system 514 can include one or more network interfaces 520 that can include, for example, an input/output (I/O) interface to external computing device 504 and/or wearable device 100. In some embodiments, server system 512 can include memory 516, and one or more communication buses for interconnecting these components.

Memory 516 according to example embodiments can include high-speed random-access memory such as, for instance, DRAM, SRAM, DDR RAM, or other random-access solid-state memory devices; and, optionally, can include non-volatile memory such as, for example, one or more magnetic disk storage devices, one or more optical disk storage devices, one or more flash memory devices, or one or more other non-volatile solid state storage devices. Memory 516 according to example embodiments, optionally, can include one or more storage devices that can be remotely located from processor(s) 518 (e.g., processing unit(s)). Memory 516 according to example embodiments, or alternatively the non-volatile memory within memory 516, can include a non-transitory computer readable storage medium. In some embodiments, memory 516, or the non-transitory computer readable storage medium of memory 516, can store one or more programs, modules, and data structures. In these embodiments, such programs, modules, and data structures can include, but not be limited to, one or more of an operating system that can include procedures for handling various basic system services and for performing hardware dependent tasks.

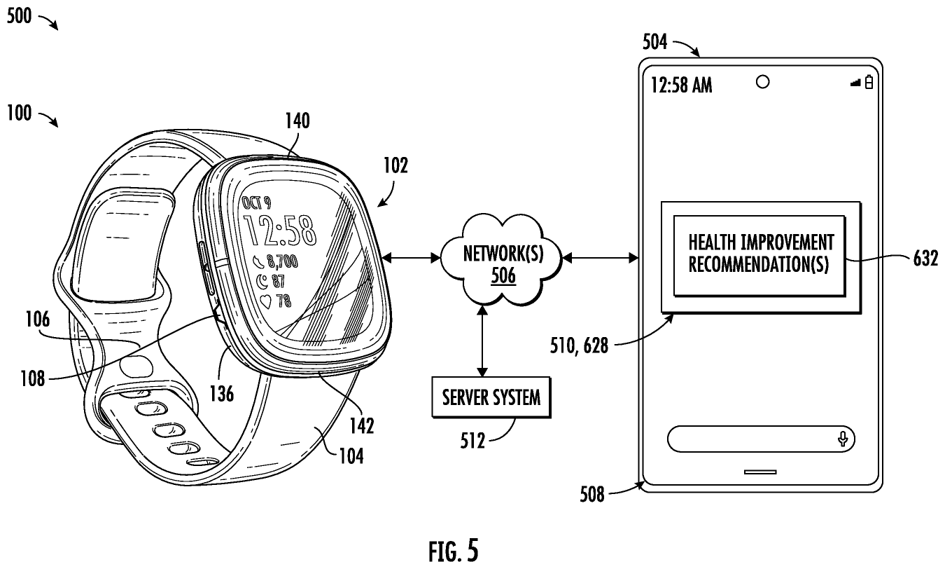

Referring now to FIGS. 7A-14, various views of a quantified-self program 600 being implemented on wearable device 100, external computing device 504, and/or server system 512 as described herein are illustrated. In particular, the quantified-self program 600 may be implemented on a computer application programmed in any of wearable device 100, external computing device 504, and/or server system 512. Accordingly, in an embodiment, the quantified-self program 600 is configured to educate users about the importance of self-experimentation, help users adopt a health behavior goal for a defined time (e.g., such as the next 28 days), allow users to report adherence to the new health behavior goal, and allow users to easily understand the magnitude and importance of change throughout the program. In addition, in an embodiment, the quantified-self program 600 may provide periodic reminders, motivational messages, and/or information about one or more metrics.

Referring particularly to FIG. 7A, as an example, the quantified-self program 600 may begin with an explanation screen 602, e.g., on display 508 of external computing device 504, that includes an explanation of the program 600 and why such experiments matter, so as to entice the user 10 to continue the program. As the user 10 continues the program 600 (e.g., by selecting a “NEXT” button 604), one or more additional initiation screens may appear to the user 10, as shown in FIGS. 7B-7D. For example, as shown in FIG. 7B, the quantified-self program 600 may display information relating to how to track success. Such information may include, for example, educating the user 10 in how to user wearable device 100 for monitoring, collecting, and tracking statistics/metrics relating to the program 600. For example, in an embodiment, the metric(s) may include heart rate variability (HRV), resting heart rate (RHR), heart rate, EDA, sleep score, restoration score, sleep duration (as well as REM %, deep sleep %, awake %), weight, body mass index (BMI), etc. Moreover, as shown in FIGS. 7C and 7D, the quantified-self program 600 may display one or more questions for the user 10 to answer relating to how the user 10 feels about the user's own health. For example, as shown, the user 10 may be asked to rate certain parameters (such as health status, stress management, energy level, activity level, sleep, nutrition, etc.), e.g., on a scale of 1 to 5, with 1 being poor and 5 being excellent. Thus, this subjective information can be used by the quantified-self program 600 to improve data collection, estimations, and/or predictions. In further embodiments, it should be understood that the subjective information may further include any other suitable information that can be input by the user 10.

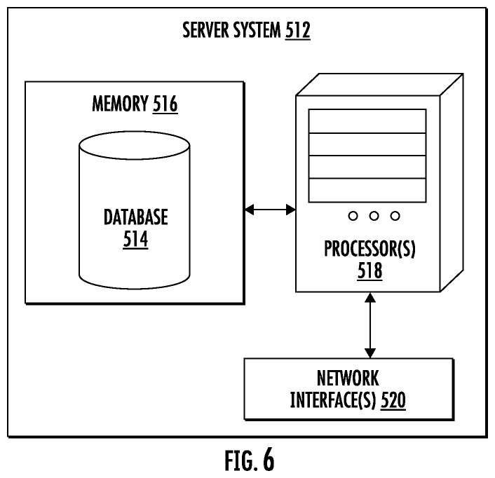

Referring now to FIGS. 8A-8D, the quantified-self program 600 is configured to prompt the user 10 to select a new health behavior goal 605 or strategy. In an embodiment, for example, the health behavior goal 605 can be manually selected by the user and may include the specific behavior or type, a measurable amount (e.g., how often and how much), attainability of the goal, relevancy (e.g., likelihood to improve one or more metrics), and/or the defined time for achieving the goal. In addition, as shown in FIGS. 8A-8D, the user 10 may select the new health behavior goal 605 from a predefined list 606. Such predefined health behavior goals may include, for example, various goals relating to fitness 608 (FIG. 8B), mindfulness 610 (FIG. 8B), sleep 610 (FIG. 8C) and/or nutrition 614 (FIG. 8D).

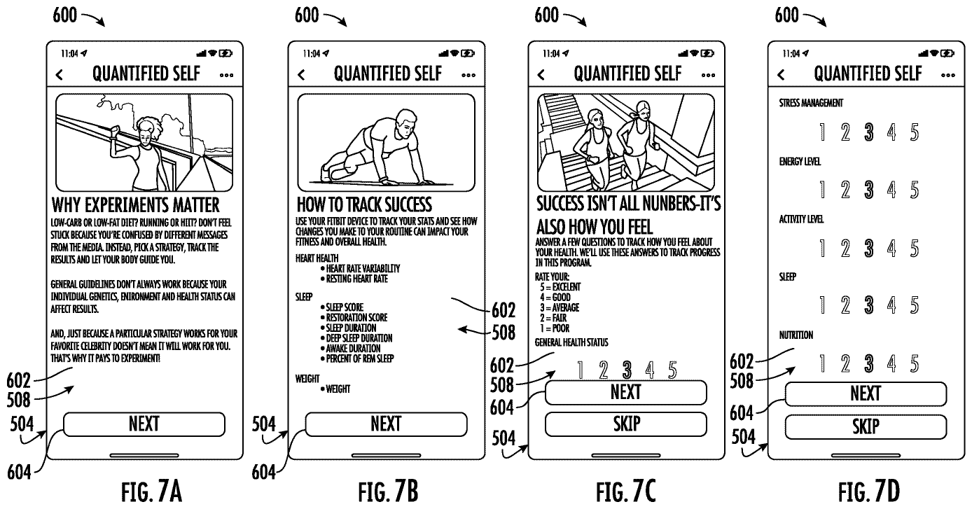

Referring now to FIG. 9, the user 10 can then be prompted to assess goal achievability 614. For example, in an embodiment, the user 10 can provide subjective information relating to whether a particular goal will be easily achieved or not. Such subjective information can be collected, for example, in the form of a rating scale (e.g., from 1 to 10, with 1 being the most difficult to achieve and 10 being very easily achievable). Once selected by the user 10, the user 10 can continue with the program 600 by selecting the NEXT button 604. In an embodiment, goal achievability 614 may be optional and skipped over by the user 10, for example, as shown via SKIP button 616.

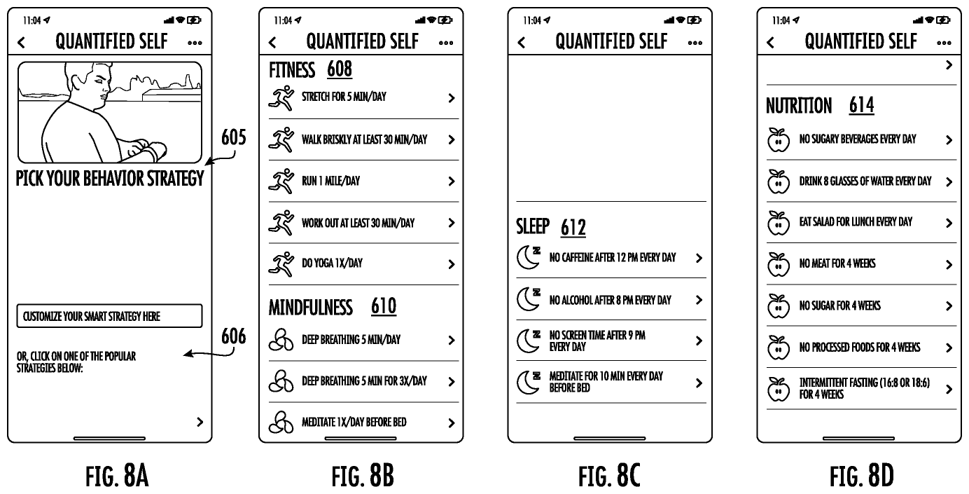

Similarly, in FIGS. 10A-10C, the user 10 may be prompted to provide additional subjective information, such as what the user 10 will do to adhere to the health behavior goal 605 (FIG. 10A), what are some potential barriers to achieving the health behavior goal 605 (FIG. 10B), and/or a reminder/notification configured by the user 10 which can be sent to the user's 10 wearable device 100, and/or external device 504 at the given time (e.g., 9:00AM) (FIG. 10C). Moreover, as shown, in an embodiment, once the information is input by the user 10, the user 10 can continue with the program 600 by selecting the NEXT button 604. In another embodiment, entering such subjective information may be optional and skipped over by the user 10, for example, as shown via SKIP button 616.

Accordingly, the quantified-self program 600 is configured to monitor or track one or more metrics (objective or subjective) relating to the user, with the metric(s) being associated with the health behavior goal 605. For example, as mentioned, in an embodiment, objective metric(s) may include HRV, RHR, heart rate, EDA, sleep score, restoration score, sleep duration (as well as REM %, deep sleep %, awake %), weight, body mass index (BMI), etc. In additional and/or alternative embodiments, the quantified-self program 600 can monitor (e.g., track) the user's physiological data over a defined period of time (e.g., 1 day, 1 week, 1 month, 3 months, 6 months, 1 year, etc.) so as to have a more accurate picture of the user's data and characteristics.

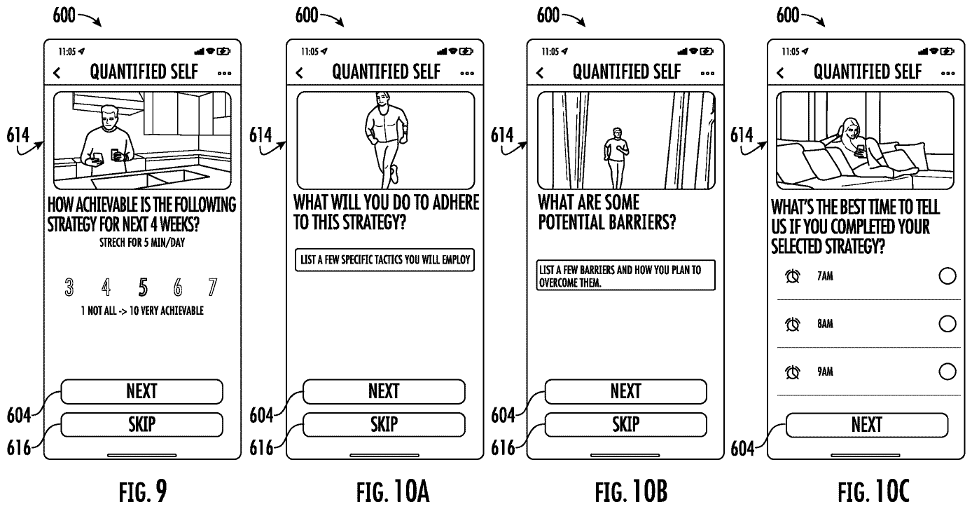

Thus, as shown in FIG. 11, while participating in the quantified-self program 600, the user 10 can easily view the tracked metric(s) associated with the health behavior goal 605 using e.g., display 508 of external computing device 504. In the illustrated embodiment, for example, display 508 may include one or more indicators of various metrics, such as steps taken, calories burned, distance walked or ran, floors climbed, and/or sleep duration. Accordingly, the user 10, by viewing the display 508, can be made easily aware of current metric(s) associated with the health behavior goal 605.

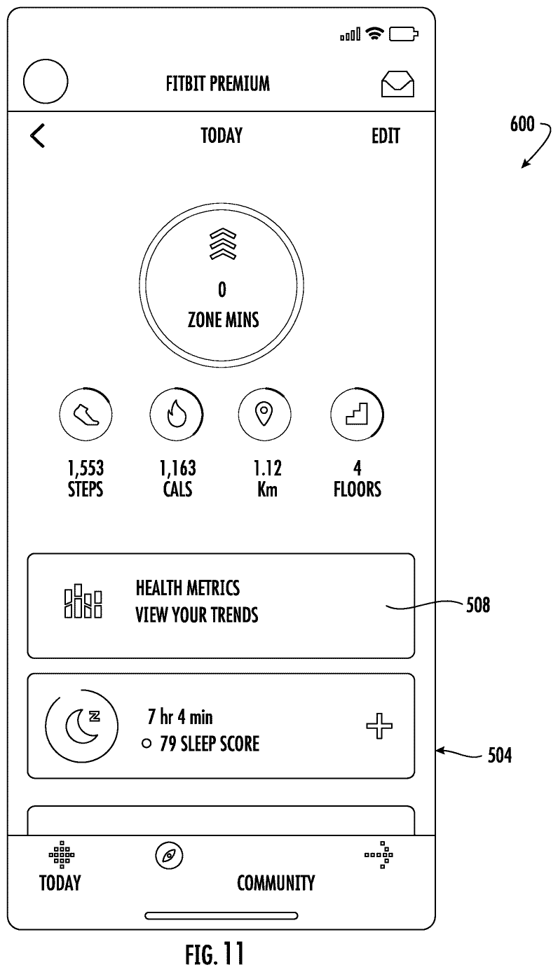

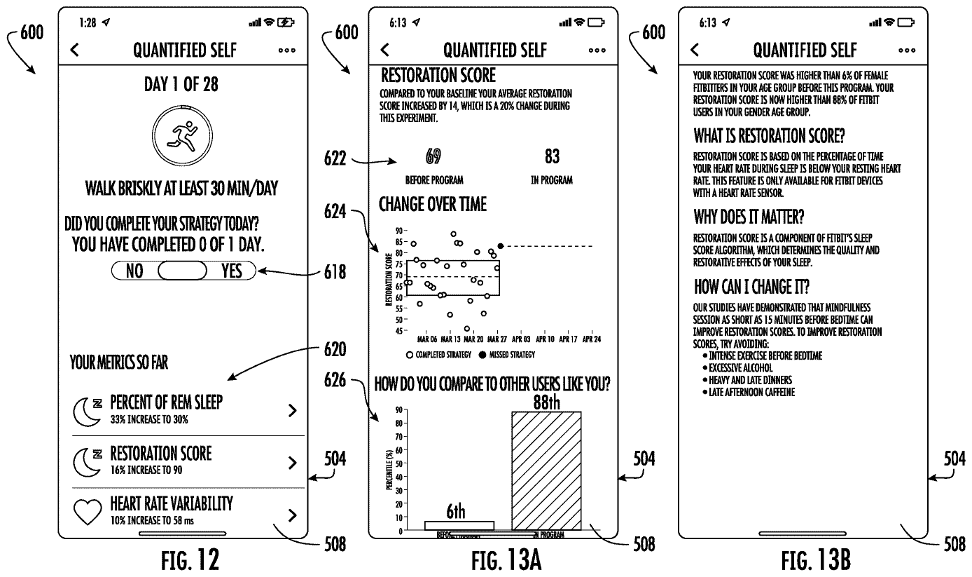

Referring now to FIGS. 12-13B, additional screens that may be displayed to the user 10 of the quantified-self program according to the present disclosure are provided. In particular embodiments, as shown in FIG. 12, the quantified-self program is configured to track adherence to the health behavior goal 605 by the user 10 over a defined time. In the illustrated embodiment, for example, the defined time may be 28 days. In other embodiments, it should be understood that the defined time may be any suitable time period including more than 28 days or less than 28 days. Further, as shown, the quantified-self program 600 is configured to prompt the user 10 to answer whether the user 10 completed the health behavior goal 605 for a particular day. Thus, as shown, in an embodiment, the user 10 can answer YES or NO using e.g., a sliding bar 618. In another embodiment, the health behavior goal 605 can be tracked automatically/objectively or manually/subjectively, depending on whether the goal can be passively and objectively tracked by wearable device 100 or external computing device 504. In addition, as shown, metrics 620 being monitored due to their association with the health behavior goal 605 can be displayed to the user 10. In particular, as shown, the health behavior goal 605 may be walking briskly at least 30 minutes per day. Thus, as shown, the displayed metrics 620 may include a percent of REM sleep, restoration score, heart rate variability, etc.

Accordingly, in an embodiment, the quantified-self program 600 is configured to detect a statistical change to at least one metric value of the metric(s) 620 based at least in part on adherence to the health behavior goal 605 by the user 10 over the defined time. As such, the quantified-self program 600 is configured to observe whether such metrics 620 change due to the user 10 working towards the health behavior goal 605. In particular embodiments, the quantified-self program 600 may be configured to detect the statistical change to the metric value(s) of the metric(s) using one or more algorithms or models (such as the ML or AI models described herein) based at least in part on adherence to the health behavior goal 605 by the user over the defined time. Furthermore, in an embodiment, the quantified-self program 600 may be configured to train the algorithm(s) and/or the model(s) based at least in part on the metric(s) of the physiological data of the user 10 collected over time such that the algorithm(s) and/or the model(s) identify the statistical change. Moreover, in an embodiment, the quantified-self program 600 is configured to receive subjective input data indicative of a subjective health change experienced by the user 10 over the defined time based at least in part on adherence to the health behavior goal 605 by the user 10 over the defined time and/or the health behavior goal 605 can be an objective goal tracked automatically. In such embodiments, the quantified-self program 600 is configured to identify a correlation or absence of correlation between the subjective health change and the statistical change based at least in part on receiving the input data.

In another embodiment, the quantified-self program 600 is configured to detect the statistical change to the metric value(s) of the metric(s) by comparing the metric value(s) to one or more benchmark metric values, respectively, corresponding to the metric(s) of the physiological data. In such embodiments, the benchmark metric value(s) may be captured over a second defined time that is prior to the defined time. In other words, the quantified-self program 600 may spend the second defined time prior to the defined time collecting metrics relating to the user 10 and learning about the user 10. Thus, when the metric value(s) are collected and compared to the benchmark metric value(s), the data will more accurately reflect the user 10 over time, rather than basing decisions on outliers and/or anomalies.

In additional embodiments, the quantified-self program 600 is configured to detect the statistical change to the metric value(s) of the metric(s) by determining whether the user 10 adhered to the health behavior goal 605 over at least a portion of the defined time. Moreover, in an embodiment, the quantified-self program 600 is configured to detect the statistical change to the metric value(s) of the metric(s) by detecting a defined change to the metric value(s) of the metric(s) based at least in part on the adherence to the health behavior goal 605 by the user 10 over the defined time. In such embodiments, the quantified-self program 600 is configured to detect the statistical change to the metric value(s) of the metric(s) by determining whether the defined change to the metric value(s) is a positive change to the metric value(s) or a negative change to the metric value(s). Furthermore, in an embodiment, the quantified-self program 600 is configured to detect the statistical change to the metric value(s) of the metric(s) by determining whether the defined change to the metric value(s) is above or below a defined statistical significance threshold value. It should be further understood that determining the statistical change to the metric value(s) of the metric(s) may further be completed using any combination of the steps and/or details described herein.

In still further embodiments, the quantified-self program 600 is configured to track engagement by the user 10 with wearable device 100 with respect to the health behavior goal 605 and determine at least one of a defined periodicity or one or more defined times to provide the user 10 with the statistical change and/or one or more insights associated with the statistical change based on tracking the engagement by the user 10 with the wearable device 100 with respect to the health behavior goal 605. Thus, in an embodiment, the quantified-self program 600 can estimate the best time to prompt the user 10 for a response (i.e., the time most likely to get a response from the user 10) based on tracking prior engagement.

Moreover, and still referring to FIGS. 12-13B, at any time during the program 600, the user 10 can select one or more of the metrics 620 on the display 508 to obtain more detailed data for viewing. Such selection may be first prompted by the quantified-self program 600 or may be initiated by the user 10. For example, in the illustrated embodiment of FIGS. 12-13B, the user 10 has selected restoration score for detailed viewing. Thus, as shown in FIGS. 13A and 13B, additional information relating the restoration score can be displayed to the user 10 via display 508. In particular, as shown at 622 in FIG. 13A, the quantified-self program 600 may display the restoration score both before and after starting the program 600. Moreover, as shown, the quantified-self program 600 may display one or more charts or graphs 624, 626, e.g., that may provide changes in the restoration score over the defined time and/or the restoration score of the user 10 as compared to other users. In additional embodiments, as shown in FIG. 13B, the quantified-self program 600 may further display educational content related to the restoration score (or any of the other metrics described herein). The educational content, for example, may include definitions, why such metrics are important, and how such metrics may be changed.

Referring now to FIG. 14, the quantified-self program 600 is configured to perform one or more operations based at least in part on detecting the statistical change. In an embodiment, for example, the quantified-self program 600 can use such insight(s) of the user's physiological data to train and/or implement an ML and/or AI model (e.g., a function, algorithm, process) that can include, but is not limited to, a classifier (e.g., nearest neighbor, random forest, support vector machine, decision tree, linear discriminant classifier, change point detection algorithm, etc.), a neural network, a convolutional neural network, a hierarchical clustering algorithm, a pairwise and/or multidimensional pairwise model, and/or another ML and/or AI model.

More specifically, in an embodiment, as shown in FIG. 14, the quantified-self program 600 is configured to generate an intelligent notification 628 having one or more insights associated with the statistical change based at least in part on comparison of the metric value(s) to one or more benchmark metric values respectively corresponding to the metric(s) of the physiological data of the user 10 and/or one or more second metrics of second physiological data of one or more second users. Further, in an embodiment, the quantified-self program 600 is configured to provide the intelligent notification 628 to the user 10, e.g., via display 508 or a second computing device. In some embodiments, the intelligent notification 628 can include one or more health improvement recommendations 632 (FIGS. 5 and 14) (e.g., a suggestion to perform a defined activity) that, if and/or when implemented by user 10, can facilitate progress towards the user's health behavior goal 605. For example, in an embodiment, the quantified-self program 600 can also ask the user 10 to input goals for the health metrics and can thus recommend activities and a level of aggressiveness to achieve the health outcome the user 10 hopes to achieve. In an embodiment, as shown in FIGS. 5 and 14, external computing device 504 can render intelligent notification 628 having the health improvement recommendation(s) on display 508 such that user 10 and/or another entity (e.g., health care professional, mental health care professional, sleep therapy provider, doctor, caregiver) can view such information.

For example, as shown particularly in FIG. 14, the quantified-self program 600 may be configured to provide a summary report 630 to the user 10 during or after the defined time. In an embodiment, for example, the quantified-self program 600 may be configured to implement one or more algorithms to determine what period of the user's time is best to use for baseline data with automatic anomaly detection, removal, etc. In another embodiment, the user 10 can select this time period. More specifically, as shown, the summary report 630 may include how many days the user 10 adhered to the program 600, along with a corresponding percentage. In addition, as shown, the summary report 630 may include various statistics relating improved or positive health metrics, unchanged health metrics, and/or declined or negative health metrics. Moreover, as shown, the summary report 630 may also include one or more recommendations 632 for the user 10. In certain embodiments, the recommendations 632 may suggest that the user 10 select one or more suggested health behavior goals based on, for example, adherence to the health behavior goal 6-4 by the user 10 over the defined time, adherence to one or more historical health behavior goals by the user 10 over one or more historical periods of time, one or more characteristics of the user 10, and/or a behavior goal selection pattern of the user 10. Similarly, the quantified-self program 600 is configured to provide the intelligent notification to at least one of the user 10 or a second computing device.

### Example Methods

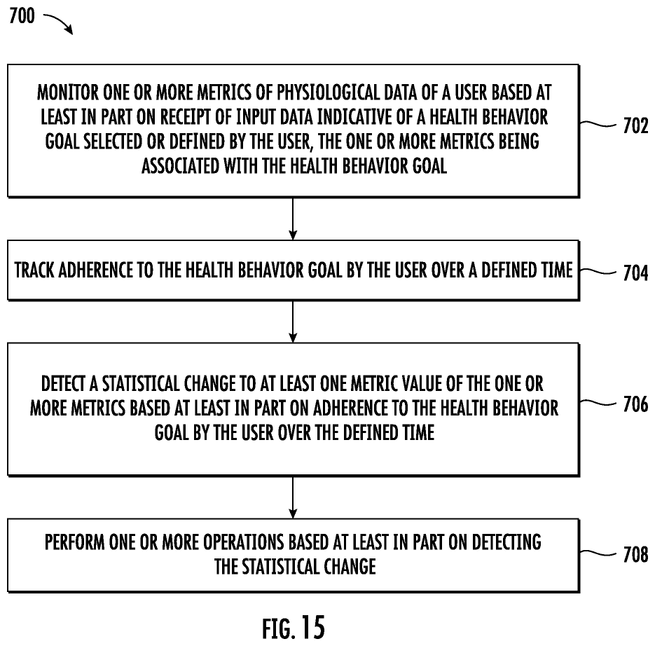

FIG. 15 illustrates a flow diagram of an example, non-limiting computer-implemented method 700 according to one or more example embodiments of the present disclosure. Computer-implemented method 700 can be implemented using, for instance, wearable device 100, external computing device 504, and/or server system 512 described above with reference to the example embodiments depicted in FIGS. 1-14.

The example embodiment illustrated in FIG. 15 depicts operations performed in a particular order for purposes of illustration and discussion. Those of ordinary skill in the art, using the disclosures provided herein, will understand that various operations or steps of computer-implemented method 700 or any of the other methods disclosed herein can be adapted, modified, rearranged, performed simultaneously, include operations not illustrated, and/or modified in various ways without deviating from the scope of the present disclosure.

At 702, computer-implemented method 700 can include monitoring, by a computing device (e.g., wearable device 100, external computing device 504, and/or server system 512) operatively coupled to one or more processors (e.g., processor(s) 181, processor(s) 518), one or more metrics of physiological data of a user based at least in part on receipt of input data indicative of a health behavior goal selected or defined by the user, the one or more metrics being associated with the health behavior goal.

At 704, computer-implemented method 700 can include tracking, by a computing device (e.g., wearable device 100, external computing device 504, and/or server system 512) operatively coupled to one or more processors (e.g., processor(s) 181, processor(s) 518), adherence to the health behavior goal by the user over a defined time.

At 706, computer-implemented method 700 can include detecting, by a computing device (e.g., wearable device 100, external computing device 504, and/or server system 512) operatively coupled to one or more processors (e.g., processor(s) 181, processor(s) 518), a statistical change to at least one metric value of the one or more metrics based at least in part on adherence to the health behavior goal by the user over the defined time.

At 708, computer-implemented method 700 can include performing, by a computing device (e.g., wearable device 100, external computing device 504, and/or server system 512) operatively coupled to one or more processors (e.g., processor(s) 181, processor(s) 518), one or more operations based at least in part on detecting the statistical change.

### Additional Disclosure

The technology discussed herein makes reference to servers, databases, software applications, and other computer-based systems, as well as actions performed by, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

What is claimed is:

1. A computing device, comprising:
one or more processors; and
one or more computer-readable media that collectively store instructions that, when executed by the one or more processors, cause the computing device to perform operations, the operations comprising:
monitoring one or more metrics of physiological data of a user based at least in part on a health behavior goal of the user, the one or more metrics being associated with the health behavior goal;
tracking adherence to the health behavior goal by the user over a defined time;
detecting a statistical change to at least one metric value of the one or more metrics based at least in part on adherence to the health behavior goal by the user over the defined time; and
performing one or more operations based at least in part on detecting the statistical change.

2. The computing device of claim 1, wherein monitoring the one or more metrics of the physiological data of the user based at least in part on the health behavior goal of the user comprises:
monitoring the one or more metrics of the physiological data of the user based at least in part on receipt of input data indicative of the health behavior goal being selected or defined by the user.

3. The computing device of claim 1, wherein detecting the statistical change to the at least one metric value of the one or more metrics based at least in part on adherence to the health behavior goal by the user over the defined time comprises:
detecting the statistical change to the at least one metric value of the one or more metrics using one or more algorithms or models based at least in part on adherence to the health behavior goal by the user over the defined time.

4. The computing device of claim 3, wherein the operations further comprise:
training the one or more algorithms or models based at least in part on the one or more metrics of the physiological data of the user collected over time such that the one or more algorithms or models identify the statistical change.

5. The computing device of claim 1, wherein detecting the statistical change to the at least one metric value of the one or more metrics based at least in part on adherence to the health behavior goal by the user over the defined time comprises:
comparing the at least one metric value to one or more benchmark metric values respectively corresponding to the one or more metrics of the physiological data, the one or more benchmark metric values being captured over a second defined time that is prior to the defined time.

6. The computing device of claim 1, wherein detecting the statistical change to the at least one metric value of the one or more metrics based at least in part on adherence to the health behavior goal by the user over the defined time comprises:
determining whether the user adhered to the health behavior goal over at least a portion of the defined time.

7. The computing device of claim 1, wherein detecting the statistical change to the at least one metric value of the one or more metrics based at least in part on adherence to the health behavior goal by the user over the defined time comprises:
detecting a defined change to the at least one metric value of the one or more metrics based at least in part on the adherence to the health behavior goal by the user over the defined time.

8. The computing device of claim 7, wherein detecting the statistical change to the at least one metric value of the one or more metrics based at least in part on adherence to the health behavior goal by the user over the defined time comprises:
determining whether the defined change to the at least one metric value is a positive change to the at least one metric value or a negative change to the at least one metric value.

9. The computing device of claim 7, wherein detecting the statistical change to the at least one metric value of the one or more metrics based at least in part on adherence to the health behavior goal by the user over the defined time comprises:
determining whether the defined change to the at least one metric value is above or below a defined statistical significance threshold value.

10. The computing device of claim 1, wherein performing the one or more operations based at least in part on detecting the statistical change comprises:
generating an intelligent notification comprising one or more insights associated with the statistical change based at least in part on comparison of the at least one metric value to one or more benchmark metric values respectively corresponding to the one or more metrics of the physiological data of the user; and
providing the intelligent notification to at least one of the user or a second computing device.

11. The computing device of claim 1, wherein performing the one or more operations based at least in part on detecting the statistical change comprises:
generating an intelligent notification comprising one or more insights associated with the statistical change based at least in part on comparison of the at least one metric value to one or more benchmark metric values respectively corresponding to one or more second metrics of second physiological data of one or more second users; and
providing the intelligent notification to at least one of the user or a second computing device.

12. The computing device of claim 1, wherein performing the one or more operations based at least in part on detecting the statistical change comprises:
generating an intelligent notification comprising a recommendation that the user select one or more suggested health behavior goals based at least in part on at least one of adherence to the health behavior goal by the user over the defined time, adherence to one or more historical health behavior goals by the user over one or more historical periods of time, one or more characteristics of the user, or a behavior goal selection pattern of the user; and
providing the intelligent notification to at least one of the user or a second computing device.

13. The computing device of claim 1, wherein the operations further comprise:
tracking engagement by the user with the computing device with respect to the health behavior goal; and
determining at least one of a defined periodicity or one or more defined times to provide the user with at least one of the statistical change or one or more insights associated with the statistical change based at least in part on tracking engagement by the user with the computing device with respect to the health behavior goal.

14. The computing device of claim 1, wherein the operations further comprise:
receiving input data indicative of a subjective health change experienced by the user over the defined time based at least in part on adherence to the health behavior goal by the user over the defined time; and
identifying a correlation or absence of correlation between the subjective health change and the statistical change based at least in part on receiving the input data.

15. A computer-implemented method, comprising:
monitoring one or more metrics of physiological data of a user based at least in part on receipt of input data indicative of a health behavior goal selected or defined by the user, the one or more metrics being associated with the health behavior goal;
tracking adherence to the health behavior goal by the user over a defined time;
detecting a statistical change to at least one metric value of the one or more metrics based at least in part on adherence to the health behavior goal by the user over the defined time; and
performing one or more operations based at least in part on detecting the statistical change.

16. The computer-implemented method of claim 15, wherein detecting the statistical change to the at least one metric value of the one or more metrics based at least in part on adherence to the health behavior goal by the user over the defined time comprises:
detecting the statistical change to the at least one metric value of the one or more metrics using one or more algorithms or models based at least in part on adherence to the health behavior goal by the user over the defined time.

17. The computer-implemented method of claim 16, wherein the operations further comprise:
training the one or more algorithms or models based at least in part on the one or more metrics of the physiological data of the user collected over time such that the one or more algorithms or models identify the statistical change.

18. The computer-implemented method of claim 15, wherein detecting the statistical change to the at least one metric value of the one or more metrics based at least in part on adherence to the health behavior goal by the user over the defined time comprises:
comparing the at least one metric value to one or more benchmark metric values, respectively, corresponding to the one or more metrics of the physiological data, the one or more benchmark metric values being captured over a second defined time that is prior to the defined time.

19. The computer-implemented method of claim 15, wherein detecting the statistical change to the at least one metric value of the one or more metrics based at least in part on adherence to the health behavior goal by the user over the defined time comprises:
determining whether the user adhered to the health behavior goal over at least a portion of the defined time.

20. The computer-implemented method of claim 15, wherein detecting the statistical change to the at least one metric value of the one or more metrics based at least in part on adherence to the health behavior goal by the user over the defined time comprises:
detecting a defined change to the at least one metric value of the one or more metrics based at least in part on the adherence to the health behavior goal by the user over the defined time, and
determining whether the defined change to the at least one metric value is a positive change to the at least one metric value or a negative change to the at least one metric value or determining whether the defined change to the at least one metric value is above or below a defined statistical significance threshold value.
